(19) United States
(12) Patent Application Publication
(10) Pub. No.: US 2023/0414568 A1
(43) Pub. Date: Dec. 28, 2023

(54) PHARMACEUTICAL COMBINATION FOR MOOD DISORDERS

(71) Applicant: INSTITUTO NACIONAL DE PSIQUIATRÍA "RAMÓN DE LA FUENTE MUÑIZ", Ciudad de México (MX)

(72) Inventors: Gloria Acacia BENÍTEZ KING, Ciudad de México (MX); Margarita Lucía DUBOCOVICH JERKOVICH, Ciudad de México (MX); Citlali TRUETA SEGOVIA, Ciudad de México (MX); Marcela VALDÉS TOVAR, Ciudad de México (MX); Brian Daniel QUERO CHÁVEZ, Ciudad de México (MX); Ana María DORANTES BARRÓN, Ciudad de México (MX); Rosa ESTRADA REYES, Ciudad de México (MX)

(73) Assignee: INSTITUTO NACIONAL DE PSIQUIATRÍA "RAMÓN DE LA FUENTE MUÑIZ", Ciudad de México (MX)

(21) Appl. No.: 18/351,665

(22) Filed: Jul. 13, 2023

Related U.S. Application Data
(63) Continuation of application No. 16/976,636, filed on Aug. 28, 2020, now abandoned.

(30) Foreign Application Priority Data
Feb. 28, 2018 (MX) MX/A/2018/002510

(51) Int. Cl. A61K 31/4045; A61P 25/24; A61K 9/00; A61K 31/13

(52) U.S. Cl. CPC A61K 31/4045; A61P 25/24; A61K 9/0019; A61K 31/13

(57) ABSTRACT

The present invention refers to a pharmaceutical composition that comprises the synergistic combination of an antagonist agent of the NMDA receptor, such as the active principle: ketamine and an agonist agent of the MT1 and MT2 melatonin receptors, as is the active principle: melatonin, which are in a pharmaceutical composition, which is indicated for the control and treatment of psychiatric diseases.

### FIELD OF THE INVENTION

The present invention relates to the technical field of the pharmaceutical industry, since its object is to provide a synergistic pharmaceutical combination that comprises an agonist agent of the MT1 and MT2 receptors, as is the active principle melatonin and an antagonist agent of the N-methyl-D-aspartate (NMDA) receptors, as is the active principle ketamine; which are administered with pharmaceutically acceptable excipients, a combination indicated for control and treatment of mood disorders. This combination of Melatonin-Ketamine has shown a better effect together than when they are administered independently, offering better benefits such as: administration of lower concentrations of the active principles than when they are administered separately, greater effectiveness and greater therapeutic potency and also a significant reduction in the probability of side effects that may occur when they are administered independently.

Additionally, methods are described for the identification of cell clusters as biomarkers to determine the neurogenic potential of various drugs that are useful in the treatment of neuropsychiatric diseases, allowing the identification of the best treatment for each patient, all this from neuronal precursors obtained by exfoliation of the nasal cavity.

### BACKGROUND

Mental health is a state of well-being determined by multiple social, environmental, biological and psychological factors, which can present conditions such as depression, anxiety, epilepsy, dementias, schizophrenia, and that can be a consequence of developmental disorders, some of which have worsened in recent years. In this sense, ensuring that the population conserves mental health, in addition to physical health, depends largely on the performance of successful public health actions, to prevent, treat and rehabilitate people who suffer from any alteration of the same.

The World Health Organization (WHO) estimates that neuropsychiatric disorders account for 28% of diseases worldwide, of which more than a third is caused by mood disorders. It is considered that by 2020 20% of the world population will have depression and this organization established that this group of diseases affects about 350 million people in the world and is the fourth cause of morbidity worldwide. However, it is anticipated that it will become the second cause in this category in the year 2030 (Diagnosis and Treatment of Depressive Disorder in Adults. Mexico: Ministry of Health; Dec. 1, 2015). In Mexico, according to data from the Ministry of Health, most of the people who suffer from it are adults. However, the population of children and adolescents suffering from a neuropsychiatric disease is on the rise. In addition, 50% of mental disorders start before 21 years of age. Currently 27% of patients are located in outpatient units and 6% in psychiatric hospitals.

### MOOD DISORDERS

Mood disorders are understood to be the spectrum of neuropsychiatric disorders characterized by persistent and prolonged changes in emotional state such as extreme sadness or extreme joy (mania). Particularly, depression is a common mental disorder characterized by the presence of sadness, loss of interest or pleasure (anhedonia), loss of energy (anergy), feelings of guilt, or lack of self-esteem, sleep or appetite disorders, feeling tired and poor concentration. It can become a chronic or recurrent condition and significantly impede performance in daily activities and the ability to cope with daily life.

Mood disorders include bipolar depression, when the depressive episode alternates with one of a manic nature; and unipolar depression, when it only has the depressive pole. Within unipolar depressive disorder, several subtypes are distinguished: major depressive episode, major depressive disorder (relapsing), dysthymic disorder, unspecified depressive disorder (premenstrual, minor, brief relapsing) and other mood disorders (due to medical illness, induced by substances, and non-specific).

Major Depressive Disorder (MDD) is characterized by the presence of different symptoms related with sadness, cognitive alterations and somatic symptoms. Children or adolescents with MDD experience low mood, which, unlike adults, manifests itself mainly with irritability, inability to enjoy things they like, concentration problems compared to previous functioning, attention failure, ideas of being less valuable than others, isolation, decreased school performance, loss or noticeable increase in appetite, emotional lability, easy crying, sleep and energy disturbances, difficulty thinking about the future and when severe may present suicidal attempts or symptoms of psychosis, such as hallucinations (somatosensory disturbances) or delusions (false beliefs). The duration of symptoms is at least two weeks and represents a significant discomfort with deterioration of daily life.

At present, depression can be treated with various medications. These include tricyclic antidepressants (imipramine, amitriptyline), tetracyclic antidepressants, monoamine oxidase inhibitors (MAOIs) (phenalzine, tramilcypromine, deprenyl), selective serotonin reuptake inhibitors (SSRIs), atypical antidepressants, lithium salts and reversible inhibitors of the Monoamine oxidase (MAO) (moclobemide, mirtazapine, nefazodone, venlafaxine).

Although there are many medications for the treatment of different types of depression, they have adverse side effects as a disadvantage, such as: tachycardia, tremor, anxiety, nausea, dry mouth, constipation, urinary retention, confusion, delirium, sedation, drowsiness and orthostatic hypotension. Likewise, they generate the antidepressant effect expected after 3 weeks of administering the treatment and sometimes the therapeutic effect is obtained after a prolonged treatment of months.

Therefore, an effective treatment is necessary to provide the expected antidepressant effect in less time and with fewer adverse effects, as well as a useful methodology to determine the neurogenic potential of drugs useful in the treatment of neuropsychiatric diseases such that it allows to define a personalized treatment for each patient.

Ketamine is the compound 2-(2-chlorophenyl)-2-(methylamino) cyclohexanone, represented by the formula (I):

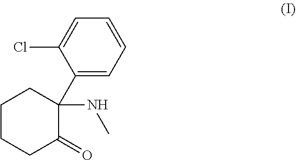 [SMILES: CNC1(c2ccccc2Cl)CCCCC1=O]

First described in U.S. Pat. No. 3,254,124 with activity in the central nervous system. It is used primarily as a dissociative anesthetic agent, providing anesthesia that includes hypnosis, powerful analgesia, and neuroendocrine protection, in addition to considerable amnesia (White PF. et. al., Ketamine-its pharmacology and therapeutics uses. 1982). It has a high bioavailability after intravenous or intramuscular administration. Its initial metabolic rate and low absorption require high doses when administered orally or rectally. This compound acts as an antagonist of NMDA receptors, so it has anti-glutamatergic actions and appears to have rapid antidepressant effects, making other treatments seem limited. In subjects with MDD and suicide victims, evidence was found indicating that glutamate levels are increased in different regions of the CNS and particularly in the prefrontal cortex.

Melatonin is the compound N-[2-(5-methoxy-1H-indol -3-yl) ethyl] acetamide, represented by the formula (II):

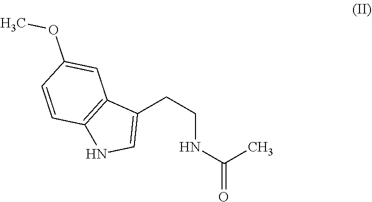 [SMILES: COc1ccc2[nH]cc(CCNC(C)=O)c2c1]

Widely known as an indole-derived hormone produced primarily at night by the pineal gland that plays an important role in regulating circadian cycles such as appetite rhythm, body temperature, and sleep-wake cycle, among others. In 1979, major depressive disorder was named as “low melatonin syndrome,” a concept focused on low melatonin secretion as a biological marker of depression.

Although the exact role of melatonin in the pathogenesis of depression is uncertain, nocturnal decreases in plasma melatonin levels have been reported both in animal models of depression and in depressed patients. Melatonin deficiency is associated with a predisposition to suffer from mood disorders, characterized by psychopathological and neurobiological alterations, including anhedonia, agitation, sleep disturbances, fluctuations in the circadian cycle, weight loss, and increased monoamine oxidase activity and plasma cortisol levels. The results of preclinical studies indicate that the combination of antidepressants with melatonin serves as an effective strategy for depression treatment. And according to studies using trials based on preclinical antidepressant behavior, it has been revealed that MT1 and MT2 melatonin receptors, are important for the development of new antidepressant drugs.

In the state of the art, a method for treating post-traumatic stress disorder (PTSD) is described, which comprises treating a human individual suffering from it with an effective therapeutic amount of ketamine by means of the intravenous or intranasal administration described in the patent application MX/a/2015/014385; a method for treating depression, which comprises administering intranasally to a patient who has not responded to at least two suitable antidepressant treatments by means of a composition comprising ketamine at a sufficient dosage described in U.S. Pat. No. 8,785,500; these patents have in common that they are focused on a treatment of psychiatric illness; a mucoadhesive delivery system comprising a partially water-soluble bioadhesive layer comprising a bioadhesive polymer or a combination of a bioadhesive polymer and a first film-forming water-soluble polymer, a partially water-soluble non-adhesive backing layer comprising a second water soluble film-forming polymer; at least one pharmaceutical agent and one mucosal penetration enhancing agent, in which the system is mucoadhesive, flexible and biodegradable, where the pharmaceutical agent can be ketamine with melatonin described in patent application PA/a/2006/001776; this patent has the use of drugs such as ketamine and melatonin to be present in a mucoadhesive system, however, it does not mention a synergistic activity between said compounds or the control and treatment of mood disorders such as major depressive disorder and treatment-resistant depression, or amounts of drugs used in other more effective pharmaceutical forms for such treatment.

The present invention is characterized in that it provides a composition that comprises the synergistic combination of an agonist agent for MT1 and MT2 receptors (melatonin) with an antagonist agent of type NMDA glutamatergic receptors (ketamine), which is indicated for the control and treatment of mood disorders such as major depressive disorder and treatment-resistant depression.

### OBJECT OF THE INVENTION

It is widely known that there are various groups of drugs that are used in the treatment of mood disorders. Among these disorders, there are those that are resistant to currently available drug treatments. In order to offer a new therapeutic option for control and treatment of mood disorders, which manages to reduce symptoms and improve the quality of life of patients, the present invention corresponds to a synergistic pharmaceutical combination that comprises an agonist agent of MT1 and MT2 receptors, (melatonin) and an antagonist of NMDA receptors, (ketamine). This combination of Melatonin-Ketamine has shown a better effect together than when they are administered independently, offering better benefits such as: administration of lower concentrations of the active principles than when they are administered separately, greater effectiveness and greater therapeutic potency and in addition, it significantly reduces the probability of side effects that may occur when they are administered independently.

An additional object of the present invention is to provide methods for the identification of cell clusters as biomarkers to determine the neurogenic potential of various drugs that are useful in the treatment of neuropsychiatric diseases such that it allows defining a personalized treatment for each patient, all this from neural precursors obtained by exfoliation of the nasal cavity.

### BRIEF DESCRIPTION OF THE FIGURES

FIG. 1. Administration scheme in cloned neuronal precursors.

FIG. 2. Representative image splicing of neuronal precursors showing spines (B), with a negative control showing lack of staining (A).

FIG. 3. Representative image of an aggregate of neuronal precursors or “cell clusters” stained with an anti-nestin antibody (A), with the antibody that recognizes the KI67 protein (B) and with an anti-doublecortin antibody (C).

FIG. 4. Scheme of the triple melatonin administration, and a single administration of ketamine followed by the forced swim test 30 minutes later.

FIG. 5. Scheme of the exposure to the light-dark cycle of the rodents subjected to the forced swim test treated with melatonin and ketamine.

### DETAILED DESCRIPTION OF THE INVENTION

The present invention provides a composition comprising the synergistic combination of an agonist agent of the MT1 and MT2 receptors with an antagonist agent of the NMDA-type glutamatergic receptors; which are formulated with pharmaceutically acceptable excipients, indicated for the control and treatment of mood disorders such as major depressive disorder and treatment-resistant depression.

The receptor agonists agents refer to a group of drugs that bind to these MT1 and MT2 receptors to elicit an action similar to that produced by the endogenous physiological substance that is produced in the pineal gland. The MT1 and MT2 melatonin receptors are two molecules of a protein nature that are made up of 350 and 362 amino acids, respectively.

The MT1 melatonin receptor is coupled to the adenylate cyclase transduction pathway through the Gq/11 and G inhibitory proteins sensitive to pertussis toxin, decreasing the amount of cAMP (cyclic adenosine monophosphate) and the activation of protein kinase A (PKA). Stimulation of the MT1 receptor also increases phosphorylation of ERK 1/2 proteins and mitogen-sensitive kinases. The MT2 melatonin receptors are also coupled to the cAMP transduction pathway through Gi protein, as well as to the phosphatidyl inositol transduction pathway and protein kinase C (PKC) activation. It has also been reported that activation of melatonin receptors in retina causes a decrease in calcium-dependent dopamine release.

The MT1 Melatonin receptors modulate neuronal activation, arterial vasoconstriction, cell proliferation in cancer cells, and reproductive and metabolic functions. Activation of MT2 melatonin receptors changes the phase of the circadian cycle of neuronal activation in the suprachiasmatic nucleus. Furthermore, it induces vasodilation and the inhibition of leukocyte migration to vascular beds. The melatonin-mediated responses elicited by activation of the MT1 and MT2 melatonin receptors depend on the circadian rhythm, as well as the duration and concentration of plasma melatonin as well as receptor sensitivity.

The circadian production of melatonin by the pineal gland is controlled by endogenous oscillators located in the suprachiasmatic nucleus (SCHN) and is regulated by the daily and seasonal changes of the photoperiod (light-dark environmental cycle). Endogenous melatonin released from the pineal gland during the night can feedback to the Central Nervous System (CNS) through the activation of the MT1 and MT2 receptors.

Said agonist of MT1 and MT2 melatonin receptors has therapeutic effects on the central nervous system, for the treatment of insomnia and circadian sleep disorders. Also, the MT1 and MT2 receptors have been considered to improve learning and memory, to stop neurodegeneration as well as for the treatment of drug addiction. Thus, it is usually used more widely in the treatment of these disorders where the agonist agent of the MT1 and MT2 receptors can be, for example, agomelatine, tasimelteon, ramelteon and melatonin or their pharmaceutically acceptable salts of the above.

According to the present invention, said MT1 and MT2 receptor agonist is contained in an amount of 1 to 300 parts in molar concentration in the preparation of the pharmaceutical combination.

In a preferred embodiment, said MT1 and MT2 receptor agonist is melatonin or a pharmaceutically acceptable salt thereof, which is administered in the pharmaceutical combination in a dose of 4 to 48 mg/Kg; by a single administration, in two administrations or in three administrations, until achieving the adequate therapeutic dose; wherein at least one of the administrations is carried out in combination with an antagonist agent of the NMDA-type glutamatergic receptors.

NMDA-type glutamatergic receptors are cellular receptors (proteins or glycoproteins) belonging to a GluN subgroup. There are also AMPA and Kainate glutamatergic receptors. All these receptors are ionotropic, they are fundamental for the development of the central nervous system (CNS), present in the neuronal synapses that participate in the regulation of the postsynaptic excitatory potential, the generation of rhythms for respiration and locomotion and have activity in neuronal plasticity, learning and memory. NMDA receptors are important in the treatment of many CNS disorders, including stroke, hypoxia, ischemia, head trauma, Huntington's, Parkinson's and Alzheimer's diseases, epilepsy, neuropathic pain, alcoholism, schizophrenia and mood disorders. To date, drugs that target NMDA receptors have had limited clinical success due to low efficacy and unacceptable side effects, including hallucinations, catatonia, ataxia, nightmares, and memory deficits. Thus, NMDA receptor antagonists have usually been used for the treatment of these disorders. The antagonist agent of the NMDA receptors can be, for example, any one of ketamine, dextromethorphan, methadone, memantine, amantadine or their pharmaceutically acceptable salts of all the above.

According to the present invention, said NMDA receptor antagonist is contained in an amount of 1 to 3333 parts in molar concentration in the preparation of the pharmaceutical combination.

In a preferred embodiment, said NMDA receptor antagonist is ketamine or a pharmaceutically acceptable salt thereof, which is administered in the pharmaceutical combination in a dose of 1 to 9 mg/Kg; by a single administration, in two administrations or in three administrations, until achieving the adequate therapeutic dose; wherein at least one of the administrations that is carried out in combination with an MT1 and MT2 receptor agonist is melatonin.

Ketamine produces a rapid antidepressant response in patients with resistant major depressive disorder that causes remission after 16 days. Despite these effects, ketamine produces undesirable psychotomimetic and dissociative effects.

The preparation of the pharmaceutical combination of the present invention comprises both active principles at the same time. It is important to select the type and quantity of excipients so that the dissolution profile of each active principle in the preparation of the combination is not affected by any interaction between melatonin and ketamine.

Ketamine does not interfere with the metabolism of other drugs that have an activity on the CNS, the action mechanism of ketamine is different from that of the agonist drugs of the MT1 and MT2 receptors.

As part of the efficiency and tolerance evaluation of the pharmaceutical combination reason for the present invention, as well as the synergistic effect of the active principles melatonin and ketamine combined, a comparative study of preclinical phase was carried out in which the active principles aforementioned were administered separately active principles, as well as the combination of these to neuronal precursor cultures to evaluate the biological effects of an antidepressant type such as neurogenesis and the formation of spines that other antidepressants induce.

Neuronal precursors are cells that are already committed to the neuronal lineage at a previous stage. Cultures of neuronal precursors obtained from the olfactory epithelium by exfoliation maintain the ability to form phenotypic structures dependent on the cytoskeleton, such as growth cones, lamellipodia, microspikes, and short or long neurites. This organization capacity of the cytoskeleton is preserved in cryopreserved, thawed and cultured cells under the same conditions as heterogeneous and selected cultures. Furthermore, neuronal precursor cells have the ability to mature spontaneously or in response to differentiation factors such as cAMP, brain-derived neurotrophic factor (BNDF) or forskolin, so mature neurons are observed in the culture obtained by exfoliation.

By means of the method described below, it was evaluated and substantiated that when the active principle ketamine is administered intraperitoneally in combination with the active principle melatonin, a more effective effect and a more powerful therapeutic activity are presented in antidepressant-type behavior in an experimental model of forced swimming than it is used to simulate depression in rodents.

This method, in addition to serving to evaluate the activity of the melatonin-ketamine combination, was effective for determining the effectiveness of fluoxetine, as well as the concentrations required for each group under evaluation, so that through an in vitro evaluation, it allows to generate the best treatment parameters, that is, dose and active principle.

### METHOD IN CLONED NEURONAL PRECURSORS

Cloned neuronal precursors obtained by exfoliation of the nasal cavity of a healthy female subject without neuropsychiatric disease at the time of sample collection were used. These were grown according to the following process:
a) Solution 1 containing the culture medium characterized by containing one or more amino acids, one or more mineral salts, one or more vitamins, one or more growth factors of natural origin, and one or more antibiotics;
b) Solution 2 containing one or more pH regulators, and one or more calcium chelating agents, whose function is to moisten the cells that will later be propagated;
c) Solution 3 containing one or more substances that break proteins and that allow the separation between the cells by dispersing them;
d) Solution 4 containing one or more substances that allow the cells to be fixed to a glass support;
e) Solution 5 of cryopreservation containing one or more cryoprotective agents of neuronal origin cells;
f) Solution 6 of differentiation containing the maintenance solution without natural origin growth factors, and a cyclic nucleotide compound that induces differentiation;
g) Solution 7 containing one or more identifiers of specific protein molecules of the neuronal structure such that they allow to distinguish the neuronal cells from patients with neuropsychiatric diseases from healthy subjects; and
h) Solution 8 containing one or more fluorescent and/or enzymatic markers to recognize the identifiers of solution 7; up to passage 5 at a temperature of 37° C. in a 5% CO² atmosphere. Cells in passage 5 were incubated with increasing concentrations of ketamine or melatonin or the combination of both according to the following administration schedules:

### In Cloned Neuronal Precursors

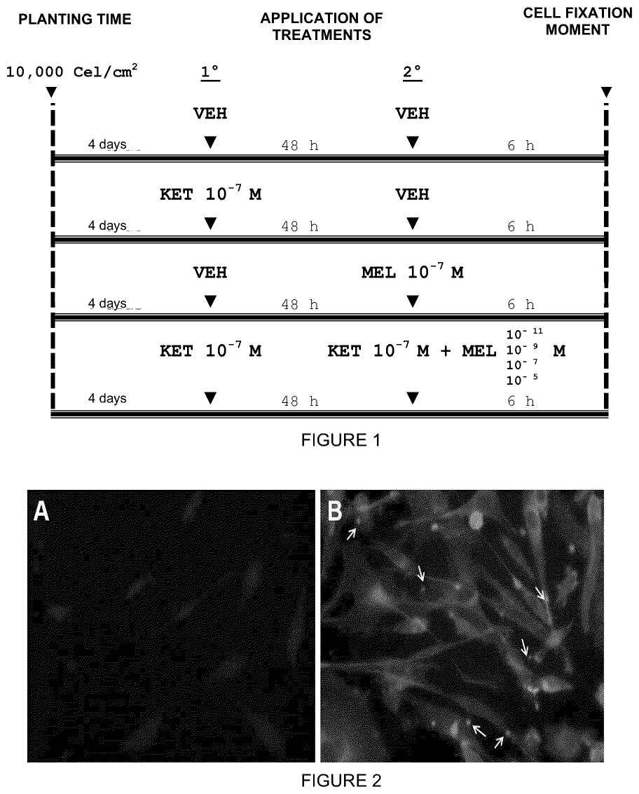

i. Incubation of the neuronal precursors with a vehicle (0) and ketamine for 12 hours (1), 24 hours (2), 48 hours (3) and 72 hours (4).
ii. Incubation of neuronal precursors with 10⁻⁷ molar ketamine for 48 hours followed by incubation for 6 hours with 10⁻⁷ molar ketamine and melatonin concentrations ranging from 10⁻¹¹ molar (7), from 10⁻⁹ molar (8), 10⁻⁷ molar (9) and 10⁻⁵ molar (10), this evaluation was incorporated in a treatment with vehicle (0), with 10⁻⁷ molar melatonin (5) and 10⁻⁷ molar ketamine (6). Said treatment is outlined in FIG. 1.
iii. The incubation of the neuronal precursors with a triple application of three vehicle administrations (11), three administrations of 10⁻⁷ molar melatonin (12), two vehicle administrations+one administration of 10⁻⁷ molar ketamine (13), three administrations of 10⁻¹¹ molar melatonin+one administration of 10⁻⁷ molar ketamine (14), three administrations of 10⁻⁹ molar melatonin+one administration of 10⁻⁷ molar ketamine (15), three administrations of 10⁻⁷ molar melatonin+one administration of 10⁻⁷ molar ketamine (16), three administrations of 10⁻⁵ molar melatonin+one administration of 10⁻⁷ molar ketamine (17). The first melatonin administration is applied and incubated for 17 hours, followed by a second application of melatonin and incubation of 7 hours, and a third application is carried out half an hour before fixing the cells, the combination of molar melatonin and ketamine is administered, except in treatments 11, 12 and 13. Said treatment is outlined in FIG. 4.

Once the cells were fixed, they were processed to stain them with specific markers that recognize the spines, which are made up of spinophilin and filamentous actin (FIG. 2A and 2B). The anti-spininophilin antibody was labeled with a second antibody having fluorescein isothiocyanate (green labeled) (FIG. 2B). Filamentous actin was labeled with rhodamined phalloidin (marked in red) (FIG. 2B). The spines were counted in 10 fields chosen at random and normalized between the total cells number present in each field (Table 1). The formation of spines corresponds to potential connection sites between neurons (synapses) as a result of the antidepressant action of the administered active principles.

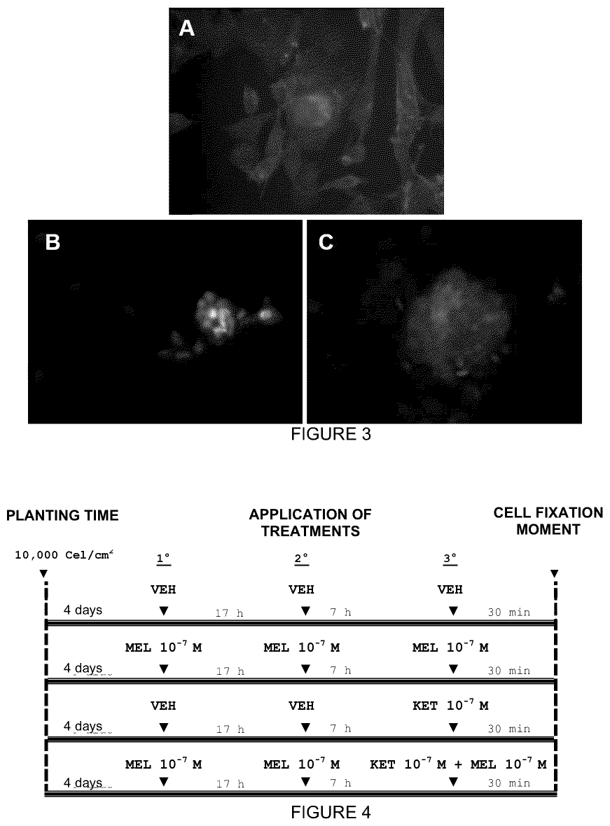

Cell groupings or “cell clusters” made up of neuronal precursors in cell division were also identified (FIG. 3A, 3B and 3C). These cell clusters were identified by staining with an anti-doublecortin antibody that is a marker of neurogenic activity (FIG. 3C), with an antibody that recognizes nestin (FIG. 3A) that is a marker of immature neuronal precursors susceptible to divide and an antibody against Ki67 (FIG. 3B) which is a proliferation marker that recognizes a nuclear protein that is expressed in cells that entered the cell cycle to divide (FIG. 3B). The number of “cell clusters” were counted in 10 fields chosen at random and normalized by the total cell number that were recognized by staining with 4′, 6-diamino-2-phenidol (DAPI) (marker of nuclei in blue). The cell clusters number was determined in the ketamine-treated cultures, with melatonin and with the combination of ketamine and melatonin (Tables 2-A and 2-B).

TABLE 1
TEMPORARY COURSE OF THE KETAMINE EFFECT
ON THE FORMATION OF SPINES IN
OLFACTORY NEURONAL PRECURSORS

| | Drug | Incubation time | Spines |
|---|---|---|---|
| Group | [M] | (h) | (%) |
| 0 | Vehicle | 0 | 8.09 ± 2.59 |
| 1 | Ketamine 10⁻⁷ | 12 | 10.25 ± 1.89 |
| 2 | Ketamine 10⁻⁷ | 24 | 22.65 ± 2.16 |
| 3 | Ketamine 10⁻⁷ | 48 | 45.31 ± 4.26 |
| 4 | Ketamine 10⁻⁷ | 72 | 45.31 ± 3.88 |

TABLE 2-A
CELL CLUSTERS FORMATION OF OLFACTORY NEURONAL
PRECURSORS PRE-TREATED WITH KETAMINE

| | Drugs | | cell cluster |
|---|---|---|---|
| Group | Pre-treatment | Administration | (%) |
| 0 | Vehicle | Vehicle | 4.38 ± 0.64 |
| 5 | Vehicle | Melatonin 10⁻⁷M | 7.61 ± 0.59 |
| 6 | Ketamine 10⁻⁷M | Vehicle | 7.12 ± 0.92 |
| 7 | Ketamine 10⁻⁷M | Ketamine 10⁻⁷M + | 7.66 ± 0.58 |
| | | Melatonin 10⁻¹¹M | |
| 8 | Ketamine 10⁻⁷M | Ketamine 10⁻⁷M + | 10.63 ± 1.02 |
| | | Melatonin 10⁻⁹M | |
| 9 | Ketamine 10⁻⁷M | Ketamine 10⁻⁷M + | 13.21 ± 1.37 |
| | | Melatonin 10⁻⁷M | |
| 10 | Ketamine 10⁻⁷M | Ketamine 10⁻⁷M + | 15.43 ± 0.89 |
| | | Melatonin 10⁻⁵M | |

TABLE 2-B
CELL CLUSTER FORMATION OF OLFACTORY
NEURONAL PRECURSORS TREATED WITH
TRIPLE MELATONIN ADMINISTRATION.

| | | | | Cell |
|---|---|---|---|---|
| | Treatments | | | cluster |
| Group | 1° | 2° | 3° | (%) |
| 11 | Vehicle | Vehicle | Vehicle | 2.6 ± 1.2 |
| 12 | Melatonin | Melatonin | Melatonin 10⁻⁷M | 2.9 ± 0.9 |
| | 10⁻⁷M | 10⁻⁷M | | |
| 13 | Vehicle | Vehicle | Ketamine 10⁻⁷M | 3.1 ± 1.0 |
| 14 | Melatonin | Melatonin | Melatonin 10⁻¹¹M + | 3.9 ± 1.0 |
| | 10⁻¹¹M | 10⁻¹¹M | Ketamine 10⁻⁷M | |
| 15 | Melatonin | Melatonin | Melatonin 10⁻⁹M + | 6.8 ± 1.0 |
| | 10⁻⁹M | 10⁻⁹M | Ketamine 10⁻⁷M | |
| 16 | Melatonin | Melatonin | Melatonin 10⁻⁷M + | 7.8 ± 1.5 |
| | 10⁻⁷M | 10⁻⁷M | Ketamine 10⁻⁷M | |
| 17 | Melatonin | Melatonin | Melatonin 10⁻⁵M + | 10.4 ± 1.3 |
| | 10⁻⁵M | 10⁻⁵M | Ketamine 10⁻⁷M | |

Although this evaluation is carried out with melatonin and ketamine, this method is applicable to other drugs known in the state of the art, for the treatment of neuropsychiatric diseases, being a technique in which can be used neuronal cells, obtained by exfoliation of the nasal cavity of a subject and through the use of specific biomarkers for the active principle of interest, optimal concentrations can be defined for each patient, such that it allows avoiding the use of doses above what is required by the patient and thus minimize the drug side or adverse effects, additionally, it is possible to choose by means of this methodology the use of one drug over another, based on how its activity is in vitro in the neuronal cells of the patient.

### ANTIDEPRESSIVE EFFECT EVALUATION IN MICE

Male mice of the “Swiss Webster” strain between 25 and 35 grams were used. All mice were placed in polyethylene boxes (8 mice per box) at a controlled temperature between 21-22° C., in a room with the dark light cycle reversed (12:12 hours, with artificial light from 10 PM to 10 A.M). The behavior evaluations of the mice were carried out between 10 AM and 3 PM. These mice were fed Purina® for rodents and water ad libitum.

A single intraperitoneal administration (I.P.) was performed in a volume of mL/kg of body weight, as the following treatment scheme:

Group 18 was administered I.P. the vehicle (saline).

Group 19 was administered I.P. fluoxetine 5 mg/kg of body weight.

Group 20 was administered I.P. fluoxetine 10 mg/kg of body weight.

Group 21 was administered I.P. melatonin 4 mg/kg (2-8 mg/Kg) of body weight.

Group 22 was administered I.P. melatonin 16 mg/kg (10.0-20.0 mg/Kg) of body weight.

The results are presented in Table 3-A.

TABLE 3-A
EFFECT OF TRIPLE ADMINISTRATION OF FLUOXETINE
AND MELATONIN ON THE IMMOBILITY TIME
OF MICE SUBJECTED TO THE FORCED SWIM TEST

| | Drugs | Immobility time |
|---|---|---|
| Group | (mg/kg) | (s) |
| 18 | Vehicle | 61.99 ± 6.15 |
| 19 | Fluoxetine 5 | 49.76 ± 4.65 |
| 20 | Fluoxetine 10 | 35.25 ± 3.88 |
| 21 | Melatonin 4 | 41.21 ± 7.29 |
| | (2.0-8.0) | |
| 22 | Melatonin 16 | 17.38 ± 2.51 |
| | (10.0-20.0) | |

### MELATONIN TRIPLE ADMINISTRATION TEST

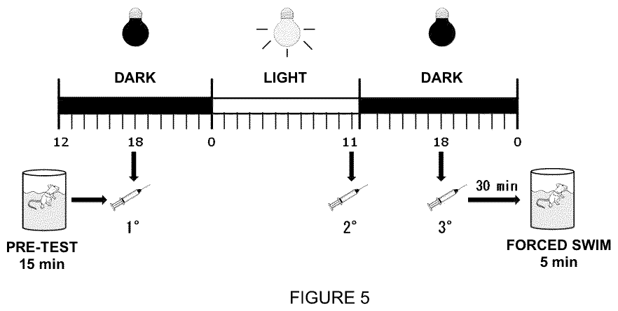

A triple intraperitoneal administration (I.P.) was performed, as in the following treatment scheme and according to what is indicated in FIG. 5:

To group 23 the first vehicle administration was done 17 hours before FST; the second vehicle administration was performed 11 hours before FST; and the third vehicle administration was performed 30 minutes before FST.

To group 24, the first melatonin administration 4 mg/kg (2.0-8.0 mg/kg) of body weight was done 17 hours before FST; the second melatonin administration 4 mg/kg (2.0-8.0 mg/kg) of body weight was carried out 11 hours before FST; and, the third melatonin administration 4 mg/kg (2.0-8.0 mg/kg) of body weight and ketamine with a sub-effective dose of 1.5 mg/kg (0.5-2.0 mg/kg) was performed 30 minutes before FST.

To group 25, the first melatonin administration 16 mg/kg (10-20 mg/kg) of body weight was made 17 hours before FST. The second melatonin administration 16 mg/kg (10-20 mg/kg) of body weight was carried out 11 hours before FST. The third melatonin administration 16 mg/kg (10-20 mg/kg) of body weight and ketamine with a sub-effective dose of 1.5 mg/kg (0.5-2.0 mg/kg) was carried out 30 minutes before FST.

The results are presented in Table 3-B.

TABLE 3-B
EFFECT OF THE TRIPLE MELATONIN ADMINISTRATION
AND A SINGLE DOSE OF KETAMINE ON THE IMMOBILITY
TIME OF MICE SUBJECTED TO THE FORCED SWIM TEST

| | Drugs | | | Immobility |
|---|---|---|---|---|
| | (mg/kg) | | | time |
| Group | 1° | 2° | 3° | (s) |
| 23 | Vehicle | Vehicle | Vehicle | 59 ± 4.41 |
| 24 | Melatonin 4 | Melatonin 4 | Ketamine 1.5 (0.5-2.0) + | 17 ± 2.11 |
| | (2.0-8.0) | (2.0-8.0) | Melatonin 4 (2.0-8.0) | |
| 25 | Melatonin 16 | Melatonin 16 | Ketamine 1.5 (0.5-2.0) + | 36 ± 3.45 |
| | (10.0-20.0) | (10.0-20.0) | Melatonin 16 (10.0-20.0) | |

### FORCED SWIM TEST (FST)

The forced swim test consists of two sessions. In the first session (pre-test), the animals are placed in a glass cylinder with the following dimensions: 20 cm in diameter by 30 cm in height. The glass cylinder is filled with water to a height of 15 cm, at 23-25° C. temperature. The mice are kept in the cylinder for 15 minutes and are naturally forced to swim. The second session takes place 24 hours after the first session and 30 minutes after administering the different pharmacological treatments. The mice are placed in the glass cylinder for 5 minutes and videotaped for further analysis. The immobility of mice in FST is defined as depressive-type behavior and is manifested by a minimal amount of movements made by the animal to stay afloat. The antidepressant effect of a drug is reflected in this test as a decrease in immobility time and an increase in swimming compared to control animals (administered with the vehicle).

### TAIL SUSPENSION TEST (TST)

The acoustically and visually isolated mice were held by the tail and suspended 50 cm above the surface of a wooden box with adhesive tape placed approximately 1 cm from the tail tip. Each mouse was suspended for a total of 6 minutes and lasting immobility was recorded for the final 4 minutes of the test. The immobility behavior was scored only when the mouse was passively hung and completely immobile.

### OPEN FIELD TEST (OFT)

In order to discard nonspecific actions of any of the treatments on locomotor activity, all the treatments studied in the TST and in the FST were analyzed in the OFT. Motor activity was measured in an apparatus formed by an opaque plexiglass box (40×30×20 cm), divided into 12 equal squares (11×11 cm). The animal was placed in a corner of the cage and its behavior was videotaped for a period of 5 minutes. An observer, who was blinded to drug treatments, recorded the total number of times the animal crossed a square during the test. A count was considered when the animal fully crossed from one plaza to the next. Any change in the number of counts is considered as an alteration in locomotor activity. The test box was carefully cleaned after each session. To avoid changes in the behavior of the animals after the first experiment, the mice were used only once per test type.

In order to explore the possible synergy effect between Melatonin and Ketamine. The following treatments were carried out:

The following groups of mice were treated under a single administration schedule: with a vehicle (26), a sub-effective dose of ketamine 1.5 mg/Kg (27), 3 mg/Kg (28), 10 mg/Kg (29), 20 mg/Kg (30), 30 mg/Kg (31), plus no effective dose of melatonin and 30 minutes later they were subjected to TST.

Under the same single administration scheme; (24, 7 and 0.5 h) before starting the trial, two other independent groups were administered with melatonin 4 mg/kg (33) and 16 mg/kg (34) plus no effective dose of ketamine, with their respective control (32). In other groups, melatonin 4 mg/kg +ketamine 1.5 mg/kg (36) and melatonin 16 mg/kg+ketamine 1.5 mg/kg (37) were administered, with their respective control (35).

The results are presented in Table 4-A.

TABLE 4-A
EFFECT OF THE SINGLE ADMINISTRATION OF KETAMINE,
MELATONIN AND THE COMBINATION, ON THE MOBILITY
OF MICE SUBJECTED TO THE OPEN FIELD TEST.

| | | Count number/ | | Stop number/ | |
|---|---|---|---|---|---|
| | Treatment | 5 min | | 5 min | |
| Group | (mg/kg) | (+/− error) | α | (+/− error) | α |
| 26 | Control | 36.87 ± 3.76 | F_{(5,47)} = 7.260, | 30.75 ± 2.57 | F_{(5,47)} = 9.797 |
| 27 | Ketamine 1.5 | 40.12 ± 3.23 | p < 0.001 | 28.62 ± 3.15 | p < 0.001 |
| | (0.5-2.0) | | | | |
| 28 | Ketamine 3 | 45.25 ± 4.29 | | 28.25 ± 2.66 | |
| | (2.5-6.0) | | | | |
| 29 | Ketamine 10 | 56.62 ± 4.36** | | 45.25 ± 2.85** | |
| | (8.0-14.0) | | | | |
| 30 | Ketamine 20 | 56.00 ± 3.62** | | 45.50 ± 3.70 | |
| | (18.0-24.0) | | | | |
| 31 | Ketamine 30 | 60.12 ± 1.60*** | | 45.37 ± 1.71 | |
| | (26-36) | | | | |
| 32 | Control | 43.87 ± 4.42 | F_{(5,47)} = 7.260 | 34.50 ± 3.95 | F_{(2,23)} = 3.295 |
| 33 | Melatonin 4 | 41.12 ± 2.48 | P < 0.001 | 25.37 ± 2.18 | p = 0.057 |
| | (2.0-8.0) | | | | |
| 34 | Melatonin 16 | 38.50 ± 3.74 | | 25.12 ± 2.36 | |
| | (10.0-20.0) | | | | |
| 35 | Control | 43.87 ± 4.42 | F_{(2,26)} = 0.248 | 34.50 ± 3.95 | F_{(2,26)} = 1.859 |
| 36 | Melatonin 4 | 40.11 ± 3.6 | p = 0.783 | 26.66 ± 2.21 | p = 0.178 |
| | (2.0-8.0) + | | | | |
| | Ketamine 1.5 | | | | |
| | (0.5-2.0) | | | | |
| 37 | Melatonin 16 | 40.90 ± 3.60 | | 27.30 ± 3.03 | |
| | (10.0-20.0) + | | | | |
| | Ketamine 1.5 | | | | |
| | (0.5-2.0) | | | | |

The following groups of mice were treated under a triple administration scheme: with a vehicle (38), three administrations of melatonin 4 mg/Kg (39), three administrations of melatonin 16 mg/Kg (40), three administrations of ketamine 1.5 mg/Kg (41), three administrations of melatonin 4 mg/Kg+one administration of ketamine 1.5 mg/Kg (42) and three administrations of melatonin 16 mg/Kg+one administration of ketamine 1.5 mg/Kg (43) and 30 minutes later they were subjected to TST.

The results are presented in Table 4-B.

TABLE 4-B
EFFECT OF THE TRIPLE ADMINISTRATION OF KETAMINE
AND MELATONIN ON THE MOBILITY OF MICE
SUBJECTED TO THE OPEN FIELD TEST.

| | | Count number/ | | Stop number/ | |
|---|---|---|---|---|---|
| | Treatment | 5 min | | 5 min | |
| Group | (mg/kg) | (+/− error) | α | (+/− error) | α |
| 38 | Control | 43.375 ± 4.71 | F_{(3,35)} = | 33.250 ± 2.72 | F _{(3,33)} = |
| 39 | Melatonin 4 | 47.200 ± 5.20 | 0.279 | 29.111 ± 2.16 | 0.865, |
| | (2.0-8.0) | | p = 0.840 | | p = 0.470 |
| 40 | Melatonin 16 | 42.625 ± 5.97 | | 26.375 ± 4.60 | |
| | (10.0-20.0) | | | | |
| 41 | Ketamine 1.5 | 41.400 ± 3.97 | | 28.000 ± 2.53 | |
| | (0.5-2.0) | | | | |
| 42 | Melatonin 4 | 36.875 ± 3.03 | F _{(2,23)} = | 28.375 ± 3.25 | F _{(2,26)} = |
| | (2.0-8.0) + | | 0.664 | | 1.859 |
| | Ketamine 1.5 | | p = 0.525 | | p = 0.178 |
| | (0.5-2.0) | | | | |
| 43 | Melatonin 16 | 37.000 ± 5.56 | | 26.750 ± 3.22 | |
| | (10.0-20.0) | | | | |
| | Ketamine 1.5 | | | | |
| | (0.5-2.0) | | | | |

### STATISTIC ANALYSIS

Data that met the normality criteria (Kolmogorov-Smirnov test) and equality of variance were analyzed using analysis of variance (ANOVA). The Bonferroni test for multiple comparisons VS control group was applied when the ANOVA showed significant difference. Values of p<0.05 were considered statistically significant.

When the data did not meet the criteria for equality, normality or variance, a non-parametric analysis was used. Differences between treated and control groups were analyzed using a Kruskal-Wallis analysis of variance in ranges (p *<0.05, p **<0.01 and p ***<0.001), followed by the Mann-Whitney rank sum test. All statistical and graphical analyzes were carried out using the Sigma Stat and Sigma Plot Software, (version 3.5, and 12.5, respectively).

### EVALUATION OF ACUTE INTRAPERITONEAL CO-ADMINISTRATION OF MELATONIN IN COMBINATION WITH KETAMINE IN THE FORCED SWIM TEST

To examine whether a melatonin and ketamine combination would be more effective than either drug alone in reducing immobility behavior, the ineffective dose of ketamine (1.5 mg/kg) (0.5 to 2.0 mg/kg) and the dose sub-effective melatonin (4 and 16 mg/Kg) (2 to 20 mg/kg) tested in previous acute experiments was selected.

With the results it was shown that a single administration with a sub-threshold dose of melatonin in combination and with an ineffective dose of ketamine under the acute scheme produced an antidepressant effect in mice.

While melatonin as ketamine (1.5 mg/kg) (0.5-2.0 mg/kg) when administered alone were ineffective, in reducing immobility time, therefore the combination significantly reduced immobility behavior compared to the control group (P<0.001**). These results indicate that the combined treatment with melatonin and ketamine produced the antidepressant-type effect in the acute treatment in mice in contrast to either of these two drugs administered alone.

### ANTIDEPRESSIVE TYPE EFFECT OF THE ACUTE TREATMENT OF THE COMBINATION OF NON-EFFECTIVE DOSES OF MELATONIN AND KETAMINE IN THE TAIL SUSPENSION TEST

As shown in FIG. 5, when the sub-effective doses of melatonin-ketamine were co-administered under the triple administration scheme, 4 or 16 mg/kg (2-20 mg/kg) of melatonin in combination with ketamine (1.5 mg/kg) (0.5-2.0 mg/kg) in the last administration, there was a significant decrease in immobility time in the TST (F (2, 31)=39.00, p<0.001), the lowest dose of melatonin (4 mg/kg)(2.0-8.0 mg/kg) was the one that produced a greater effect compared to the control group.

As shown in Table 4, the treatment with ketamine at (1.5, 3.0, 10.0 and 20.0 mg/kg), in the range of 0.5 -25 mg/kg did not produce changes in the ambulatory activity of the experimental animals subjected to OFT (F (2, 23)=0.545, p=0.589). Considering that only the highest doses of ketamine (10 30 mg/kg) modified ambulatory activity, but this did not reach statistical significance F (3, 31)=2.99, df=3, p=0.047; (Table 4A).

Regarding the tail suspension test, this indicates the effective doses of the treatment with melatonin and ketamine, either individually or in combined administration (Table 5).

TABLE 5
EFFECTIVE DOSES IN MICE IN
THE TAIL SUSPENSION TEST

| Treatment | | Count Number/5 min ± |
|---|---|---|
| (mg/kg) | N | mean standard error |
| CONTROL | 8 | 43.875 ± 4.422 |
| Melatonin 4 | 8 | 41.125 ± 2.489 |
| Melatonin 16 | 8 | 38.500 ± 3.746 |
| H = 1.059, df = 2, p = 0.589 | | |
| F_{(2,23)} = 0.545, p = 0.58 | | |
| CONTROL | 8 | 43.87 ± 4.42 |
| Ketamine 1.5 | 8 | 40.12 ± 3.23 |
| Ketamine 3 | 8 | 45.25 ± 4.29 |
| Ketamine 10 | 8 | 56.34 ± 4.36 |

H = 1.71, df = = 3, p = 0.071
F_{(3,31)} = 2.99, df = 3, p = 0.047

Evaluating the effect of melatonin in combination with ketamine on the ambulatory activity of mice in the open field test (OFT) all results are expressed as the mean±SEM of 8-12 animals. Comparisons were made using a one-way ANOVA analysis, followed by the Bonferroni test: * p<0.05, ** p<0.01, *** p<0.001.

In the current state of the art, there are pharmacological treatments for psychiatric diseases, however there is no specific treatment for mood disorders and they also do not include the ketamine with melatonin combination, which leads to non-specific treatments that last a long time, therefore the development of the present invention provides a real and safe alternative for the treatment of mood disorders, achieving a reduction in treatment times, therapeutic effects and secondary reactions.

### EXAMPLES

Some pharmaceutical compositions are described in a non-limiting manner by way of example below:

### Example 1: General Composition for the Melatonin-Ketamine Combination

| Component | Concentration |
|---|---|
| Ketamine | 1-3333 parts |
| Melatonin | 1-300 parts |
| Excipient and/or vehicle | qsp |
| pharmaceutically acceptable | |

### Example 2: Dose Composition for the Melatonin-Ketamine Combination

| Component | Dose |
|---|---|
| Ketamine | 1-9 mg/Kg |
| Melatonin | 4-48 mg/Kg |
| Excipient and/or vehicle | qsp |
| pharmaceutically acceptable | |

### Example 3: Specific Composition for the Melatonin-Ketamine Combination

| Component | Concentration |
|---|---|
| Ketamine | 0.5-2.0 mg/Kg |
| Melatonin | 2.0-8.0 mg/Kg |
| Excipient and/or vehicle | qsp |
| pharmaceutically acceptable | |

### Example 4: Injectable Solution for the Melatonin-Ketamine Combination

| Component | Concentration |
|---|---|
| Ketamine | 1-9 mg/Kg |
| Melatonin | 4-48 mg/Kg |
| Saline | qsp |

### Example 5: Oral Composition for the Melatonin-Ketamine Combination

| Component | Concentration |
|---|---|
| Ketamine | 1-3333 parts |
| Melatonin | 1-300 parts |
| Pharmaceutically acceptable | qsp |
| excipient | |

1. An in vitro method to determine the optimal and personalized concentration of melatonin and ketamine that a patient requires for the treatment of a neuropsychiatric disease, the in vitro method comprising:
i) obtain a non-invasive sample of neuronal cells by exfoliation of a subject's nasal cavity;
ii) culture the sample of neuronal cells of the step i) using a series of solutions for obtaining a clone and/or primary cultures;
iii) incubate the clone and/or the primary cultures of the step ii) in only one concentration of ketamine and in increasing concentrations of melatonin, wherein the ketamine concentration is 10⁻⁷ M and the increasing concentrations of melatonin are in the range of 10⁻¹¹ M to 10⁻¹¹ M;
iv) stain the incubating cells of the clone and/or the primary cultures of step iii) with specific biomarkers, wherein the specific biomarkers are selected from labeled antibodies for the detection of spinophylline; labeled antibodies or phalloidin-rhodamine for detection of filamentous actin; anti-doublecortin antibodies for detection of cell clusters; antibodies against nestin; and antibodies against Ki67;
v) observe the induced cellular response of the stained cells through the formation of spines and/or cell clusters; and
vi) determine the optimal and personalized concentration of ketamine and melatonin for the patient from the observed induced cellular response, wherein the optimal and personalized concentration of ketamine and melatonin is determined from the values of ketamine and melatonin concentrations that caused the appearance of spikes and/or cell clusters in the incubated cells that were observed in step v), the values of the concentrations of ketamine and melatonin used in said incubated cells being the optimal concentrations of ketamine and melatonin that must be achieved at the plasma level after administration of the such optimal and personalized concentration of ketamine and melatonin to the patient for the treatment of the neuropsychiatric disease, and wherein said optimal and personalized concentrations of ketamine and melatonin prevent or reduce psychotomimetic and dissociative adverse effects to the patient.

2. The in vitro method of claim 1, wherein the neuropsychiatric disease is a mood disorder.

3. The in vitro method of claim 2, wherein the mood disorder is depression.

4. The in vitro method of claim 1, wherein the series of solutions used in the step ii) comprises:
a. a solution 1, comprises a culture medium, amino acids, mineral salts, vitamins, growth factors of natural origin, and antibiotics;
b. a solution 2, comprises one or more pH regulators, and calcium chelating agents; whose function is to wet the cells that will be propagated later;
c. a solution 3, comprises one or more substances that break down proteins and allow separation between cells by dispersing them;
d. a solution 4, comprises one or more substances that allow the cells to be fixed to glass support;
e. a solution 5 of cryopreservation, comprising one or more cryoprotective agents from cells of neuronal origin;
f a solution 6 of differentiation, comprising a maintenance solution without growth factors of natural origin, and a cyclic nucleotide compound that induces differentiation;
g. a solution 7, comprises one or more protein molecule identifiers specific to neuronal structure such that neuronal cells from patients with neuropsychiatric diseases can be distinguished from healthy subjects;
h. a solution 8, comprising one or more fluorescent and/or enzymatic markers for recognizing the identifiers of solution 7, until passage 5 at a temperature of 37° C., in a 5% CO₂ atmosphere.
